(19) United States
(12) Patent Application Publication
(10) Pub. No.: US 2013/0184557 A1
(43) Pub. Date: Jul. 18, 2013

(54) MEDICAL FINDING SYSTEM WITH CONTROL MODULE FOR IMAGE ACQUISITION

(71) Applicants: Karlheinz Glaser-Seidnitzer, Fuerth (DE); Klaus Mayer, Eckental (DE); Mike Mueller, Moehrendorf (DE)

(72) Inventors: Karlheinz Glaser-Seidnitzer, Fuerth (DE); Klaus Mayer, Eckental (DE); Mike Mueller, Moehrendorf (DE)

(21) Appl. No.: 13/743,559

(22) Filed: Jan. 17, 2013

(30) Foreign Application Priority Data
Jan. 17, 2012 (DE) 102012200593.5

(51) Int. Cl. A61B 5/00; A61B 6/03; A61B 6/00; A61B 5/055

(52) U.S. Cl. CPC A61B 5/0013; A61B 5/055; A61B 6/03; A61B 5/7278; A61B 5/742; A61B 5/7475; A61B 6/54; A61B 6/461; A61B 6/467; A61B 6/5205; A61B 6/563
USPC 600/407

(57) ABSTRACT

In a finding system, a control system and a computer-implemented method to assess medical images, within the scope of a finding workflow a control module can be activated via which the user can enter control data at the finding system. The control data are automatically converted into control commands, which are transmitted to the acquisition system for execution. The acquisition system is expanded by an expansion module in order to transmit process data and status data to the finding system.

### BACKGROUND OF THE INVENTION

1. Field of the Invention

The present invention is in the field of medical technology and information technology, and in particular concerns the control of image acquisition processes of imaging modalities, for example magnetic resonance tomography systems, computed tomography systems or other such systems.

2. Description of the Prior Art

Medical imaging systems in modern clinical facilities normally include two spatially and structurally separated units: an acquisition system and a medical finding system. The core of the acquisition system is the image acquisition device (thus the MR scanner or data acquisition units of other imaging systems) and is computer-based. The finding system is likewise computer-based and is normally located at a distance from the acquisition system. The acquisition system typically has a console (a computer-based workstation) via which the imaging system (the MR system, for example) is controlled. A radiologist who can access the acquired image data sets via a network works at the finding workstation. Although the radiologist can access the acquired image data from his or her computer-based workstation (normally located in a radiology department or in a radiology practice of a physician in private practice) with the assistance of a picture archiving and communications system (shortened to PACS in the following), it has conventionally been possible to engage the image acquisition only to a limited extent.

However, in practice in the scope of the finding procedures, it is frequently very valuable for the radiologist to have certain capabilities to influence the acquisition system. For example, if the radiologist establishes that an image is not usable for the finding, for example because the patient has moved and the image is therefore blurry, or because the ROI region (ROI: region of interest) is only partially shown, it thus becomes necessary to request an additional examination.

For this purpose, it has conventionally been necessary for the radiologist to work with the medical technology assistant who operates the acquisition console in order to control the examination. In large clinics, the two workstations (that of the radiologist at the finding system and that of the MTA at the acquisition system) are situated far from one another, such that in the above case a telephone is normally used. Furthermore, the characterizing data must be transmitted explicitly (Which patient is it? What image sequence? Which image, specifically etc.?). Since the appertaining image is normally no longer displayed at the acquisition system (for example in an application for graphical slice positioning (GSP)) at the respective point in time of the call), the medical technology assistant at the acquisition system must establish the connection between the image to be corrected and the respective program step in what is known as a measurement queue. This previous procedure is very error-prone (for example due to incorrect associations) and time-intensive.

In the prior art it is known to provide a remote access to the acquisition system. The system “syngo Expert-i”, which is commercially available from Siemens Healthcare Systems, Inc., enables expanded access capabilities to the acquisition system from remote instances. The “syngo Expert-i” system is a computer-based product from the company Siemens AG that allows the progress of MR examinations to be obtained in real time from remote workstations. It is therefore necessary for a remote user to log into the system via specific authorization measures, normally via the input of an identification number and a password. Via a network access it is possible for the user to switch to the desktop of the acquisition system via remote control. The user, thus has the complete functionality of the acquisition system and can also execute effective and extensive measures, for example terminate an acquisition process or restart the acquisition system. It is therefore necessary to provide special authorization measures in order to be able to satisfy security requirements for a remote access. In such a system, the acquisition system and the finding system continue to be decoupled, so a remote access to the full scope of the acquisition system is provided. A remote workstation thus can join the application of the acquisition system in order to be able to observe the acquisition process.

However, even if the flexibility of the image acquisition can be increased with the syngo Expert-i system, the problem with which the assessor is confronted in his or her practical work is therefore not solved; namely, when the assessor needs to optimally quickly and simply find a solution for when the assessor must deal with unusable images within the scope of his or her finding work. The possibility that syngo Expert-i offers—namely to switch remotely to the acquisition system—proves to be unusable in practice in the present case since this tool is much too comprehensive. The radiologist does not require an access to the full scope of the acquisition system and also would not like to execute time-consuming additional authorization measures (enter a password, etc.); rather, the assessor would merely like to simply and quickly send specific control commands to the acquisition system in order to have the unusable images available with a better quality.

### SUMMARY OF THE INVENTION

An object of the present invention is to improve a computer-based system, and in particular to extend it via a control module. The extended finding system should enable control commands at the acquisition system for repeated or additional execution of acquisition processes without additional measures (authorization measures, for instance). Furthermore, a simple, efficient and flexible control of the acquisition process from the workflow of the finding should be possible.

The object is achieved in accordance with the invention by a finding system and a control system and a method and a non-transitory, computer-readable data storage medium encoded with programming instructions. The functional features of the method are embodied in corresponding objective computer-implemented modules, in particular microprocessor modules of the system. The finding system and the control system can also be integrated into the acquisition system as embedded systems.

According to one aspect of the invention, the object achievement relates to a computer-based finding system for the assessment of medical images or image data, that includes a finding computer with a monitor unit to present the image data, wherein the image data are acquired by a remote acquisition system and imported via an interface (in particular a DICOM network). The finding system is characterized by an extension in which a control module is additionally provided. The control module can be activated within the scope of the finding procedure or finding workflow and is furthermore designed to locally register control data entered at the finding system and to convert the control data automatically into control commands. The control commands are designed to be transferred directly and automatically via an interface to the acquisition system and be executed there. The control commands serve to control the image acquisition. Furthermore, the control module can additionally be designed to receive, display and/or further process status data with regard to the execution of the control commands at the acquisition system and/or with regard to the acquisition as a whole. The status data are sent from the acquisition system to the finding system via the interface.

In the following the terms used within the scope of this application are explained in detail.

The finding system is a computer workstation of the radiologist. The radiologist typically works in a radiology department that is located remote from the imaging apparatus. The finding system is thus in principle (except for the network connection) decoupled from the acquisition system. The assessment of the image data acquired by means of the acquisition system takes place at a separate, specific workstation of the radiologist after the images have been transferred to the respective computer via an interface. A client of a radiology finding software is normally installed on the finding computer. According to a preferred embodiment, it is a client of the syngo.via client/server systems. This system is designed for viewing, analysis or, respectively, evaluation and storage of the medical images.

If the radiologist establishes at the finding system that the images to be transferred are worthless for the finding, it is thus necessary to repeat corresponding measurements in order to provide the images in better quality and be able to view them. For this purpose, a function known as a repeat function must be executed at the acquisition system, this repeat function serving to repeat the respective measurement (normally with the same settings and parameters). Furthermore, it can prove to be necessary to execute a function known as an append function at the acquisition system. The append command serves to execute additional measurements, for example in the event that the transferred image data are not sufficient in order to be able to conclusively clarify a finding. Within the scope of an efficient and less error-prone finding, according to the invention it is now possible to retrieve specific control commands—in particular a repeat function and/or an append function—immediately and directly from the finding workstation and without additional authorization measures (via input of passwords or switching to additional software instances, for example) being necessary and without involvement of additional communication channels (for example telephone, fax etc.).

The acquisition system is also a computer-based workstation that is normally associated with the imaging modality. A medical technology assistant typically works at a computer at the acquisition system in order to control the implementation of the MR examination or, respectively, MR measurement. However, the invention is not limited to MR examinations; rather, it can also be applied to other imaging modalities. Upon application of the control system according to the invention to MR measurements, a special software module is provided in order to display individual measurements (image acquisitions) in what is known as a measurement queue on a monitor. As soon as the respective images have been acquired, they are likewise displayed on the monitor (typically in a separate window for a graphical slice positioning). The further examination can be planned and controlled at the scanner workstation with the data presented in such a manner. A significant advantage of the present invention is visible in that, within the scope of the finding workflow, the radiologist at his finding workstation can directly engage in the image acquisition at the scanner workstation via corresponding control commands without additional software modules needing to be implemented and activated. This advantageously leads to a marked time savings and even cost savings within the scope of the implementation of MR measurements.

The interface is a computer-based interface for a data exchange between finding system and acquisition system. Additional computer-based instances are typically connected via a network with the system mentioned in the preceding. These also interact via the same interface. The interface is advantageously a DICOM protocol (DICOM: Digital Information and Communications in Medicine). However, the invention is not limited to such a protocol and can, for example, alternatively include other network protocols (for example Internet-based protocols such as http/ip or the like). The interface is designed for the exchange of image data, control commands and/or status data. The type of data transfer is not limited in principle. However, it is normally provided that the image data, control commands and/or status data are transmitted as separate messages via the interface. Alternatively, they can also be bundled and transmitted in combination in a common packet (as a message packet).

The control module is a computer-based module. It can be fashioned as a software module or as a hardware module (as a module of a microprocessor). The control module serves to expand the finding system. The control module is integrated directly into the finding system and can also be provided at the finding system as an embedded system. In an alternative embodiment, the control module is not directly integrated into the finding system but rather is provided as a separate instance. The control module can then be executed at a separate computer-based instance that, for example, can be connected to the finding system via the interface.

The control data are registered locally at the finding system within the scope of the finding workflow. The control data are input to a user interface of said finding system. The control data are transferred automatically by the control module into control commands. The transference can include a conversion into other data formats and other command sequences. Moreover, the conversion can also include an adaptation to a different computer platform (different operating system, for example). The control data advantageously relate to a repeated image acquisition with the same measurement parameters and/or measurement commands or with different measurement parameters/measurement commands. Moreover, the control data can also relate to an additional execution of image acquisitions, for example in order to request additional image data.

The control commands can be imported directly from the acquisition system and serve to control the image acquisition of the MR scanner. The control commands are therefore acquisition system-specific.

The status data are data that characterize a status of the acquisition process. The status data comprise identification data for the respective image, currently executed measurement sequences at the acquisition system, possible error messages, time-related aspects (comprising a duration of the previously executed measurement steps, a planned duration to execute future measurement steps etc., for example). In more complex developments, the status data comprise additional metadata in relation to the execution of the image acquisition at the acquisition system, for example the underlying computer platform (operating system, small server system etc.).

A significant advantage of the invention is that the control module is directly integrated into the finding workflow. The control data can be input and registered locally (thus at the finding system) and then be translated automatically into control commands that can be executed immediately at the acquisition system. Advantageously, no additional authorization measures are required for control to be executed by the control module.

In the preferred embodiment, the control commands of the control module arrive immediately (without an intermediate step or additional user inputs) and automatically at the acquisition system for execution. In other words, the assessor can immediately execute the corresponding control commands from his or her finding workflow without needing to be authorized again at external systems, for example (via authentication measures, log-in, password input or the like, for example). No confirmation signal on the part of the acquisition system (or its users) is thus required in order to activate or, respectively, trigger the execution of the command of the control module.

However, some applications (in particular for emergency patients) require a different workflow. Therefore, in an alternative embodiment of the invention it is provided to detect a verification signal at the acquisition system. In this embodiment of the invention, the acquisition system requires a verification signal in the event that a control command that has been transmitted from the control module should be executed at the acquisition system. For example, here it can be provided that the user at the acquisition system (for example the MTA on site) enters a confirmation signal via a user interface, which identifies that the control command that has been transmitted from the control module to the acquisition system can be executed. For example, it can thus be ensured that safety-critical measurements (for instance within the scope of an emergency procedure) are not interrupted. However, this feature is merely optional and not absolutely necessary.

It is essential that the embodiment of the control module can be activated immediately, directly and automatically from the finding workflow without additional, separate registration measures (for other software applications, for instance) needing to be executed.

According to one aspect of the invention, the embodiment of the control commands (at the acquisition system) is decoupled, at least with regard to time, from the transmission of the control commands of the control module (from the finding system to the acquisition system) and/or from the receipt of the transmitted control commands at the acquisition system. An additional flexibility can therefore be achieved, for instance in that a safety-critical MR measurement is not interrupted by the receipt of control commands of the control module. In this case the control commands are written into a queue and executed at a later point in time.

By converting the control data into control commands, it is advantageously possible to implement the control module independently of the respective acquisition system. Acquisition systems that are based on different platforms (for example different operating systems, different input interfaces etc.) can therefore also be operated via one and the same control module. A mapping table in which an association between control data and control commands is stored is advantageously accessed for the conversion of the control data into control commands. Upon changing the acquisition system, only the mapping table must be adapted. The control module can therefore be designed to be manufacturer-independent (in particular independent of the manufacturer of the acquisition scanner). The additional technical effect that is connected with the conversion of the control data into control commands is visible in that an interface independence can additionally be achieved. For example, in the event that the underlying protocol requires a defined message type, this requirement can be satisfied via the conversion according to the invention (and in fact without needing to re-implement and recompile the control module). Association rules that translate the control data into control commands conforming to the interface can be stored in an additional mapping table.

According to a further aspect of the invention, the control commands include two types of commands:

1. An append command that serves to initiate an additional MR measurement, and

2. A repeat command that is designed to repeat an already executed MR measurement again.

In the case of the repeat command, the repeated measurement can be executed with the same measurement parameters in the event that—for example—a poor image quality has been caused due to the movement of the patient. Furthermore, the repeated MR measurement can be executed with different MR measurement parameters. For example, this is possible when the radiologist realizes that the images will be acquired better with different measurement parameters in order to optimize an image quality (which can ultimately increase the finding quality).

According to a further embodiment, the control commands can include additional commands that are designed to control the image acquisition. These pertain to, among other things, setting possibilities of physical measurement parameters (that can affect the image quality, among other things). The control commands also can include time specifications (for example longer or modified MR measurement sequences). Furthermore, it is possible to initiate a modified order of measurement sequences or different measurement commands (for example: a change of the radio-frequency excitation measurement pulses, such as the spin echo sequence, the gradient echo sequence or the relaxation times T1, T2). For example, the modified control commands can also include an instruction to the patient to hold his or her breath for a longer point in time in order to avoid movement artifacts.

The status data are transmitted from the acquisition system to the finding system. The status data relate to the execution of the MR measurement. The status data normally relate to a point in time after sending the control commands of the control module to the acquisition system. The finding system is informed about the current state of the measurement or, respectively, image acquisition with the status data. For example, the user is informed that his repeat command or append command is now immediately about to be executed, is presently being executed, or has been partially or entirely executed. In the preferred embodiment, following the acquisition process the newly acquired image data are transmitted immediately from the acquisition system to the finding system for a new assessment. With the status data the user at the finding system can estimate when, approximately, he can expect the modified image data.

According to a further embodiment of the invention, in addition to the status data process data are additionally transmitted from the acquisition system to the finding system. The process data are likewise electronic data in digital form that relate to the acquisition process. In principle, the process data are independent of the control commands of the control module. The process data relate to the execution of the current acquisition process on which the image data of the current finding process are based. It is possible to combine multiple process data into one message or into a message packet and then transmit them. In a preferred embodiment, the process data comprise the name of the patient, the identification number of the patient, the type of examination (for example brain scan, whole-body scan etc.) and can moreover pertain to additional details of the MR measurement (for example applied measurement sequence(s), duration, type of the acquisition system etc.). With this feature it is possible for the assessing radiologist who views the radiological image data at his or her finding workstation to be informed of the context in which the respective image data have been acquired at the acquisition system. In a preferred embodiment, the process data are considered in addition to the control data in order to generate the control commands. For example, pre-settings can be configured that are stored in a memory. These pre-settings pertain to optimization measures with regard to the image quality. Given a reduced image quality, for example, optimization settings can be adjusted with regard to the acquisition control process. From the process data it can then be derived what real values have been used in the acquisition, and from the stored optimization data from the memory it can be derived which desired values should actually have been used. The control command can then be instructions that initiate a new MR measurement with the desired parameters.

In the preferred embodiment the control data are entered directly via the interface of the finding system. In other words, it is not necessary to start an additional application since the control module is integrated into the finding system.

An additional achievement of the object exists in a control system for an MR system or other imaging apparatuses. The control system includes:
at least one acquisition system,
at least one finding system,
an interface for data exchange between acquisition system and finding system,
a control module that is respectively integrated into the finding system and is designed to register control data at the finding system and automatically convert them into control commands.

The control commands that are converted in such a manner are subsequently directly and automatically transferred via the interface to the acquisition system for execution at the acquisition system, without additional authorization measures. Furthermore, according to one embodiment the acquisition system is expanded with an expansion unit that is designed to send status data and/or process data from the acquisition system to the finding system. The status data and/or process data are displayed and/or processed further at the finding system (in particular to generate control commands).

The object also is achieved by a computer-implemented method for assessing image data. The method includes the following method steps:
First, the image data that have been acquired by the acquisition system are presented at a finding computer of the finding system.
Then possible control data are registered locally at a finding interface of the finding system. The control data characterize requirements with regard to a modified and/or expanded acquisition of the image data for the finding process.
After control data have been registered, these are automatically converted into control commands.
The generated control commands are transferred to the acquisition system (to control the measurement) automatically and without execution of authorization processes.

Depending on the embodiment, the transmitted control commands can be applied automatically at the acquisition system. The image acquisition is then controlled with the control commands of the control module without user inputs (control commands) at the acquisition system being necessary. Optionally, the application of the control commands can also be made dependent on a validation signal. Furthermore, the control commands can be executed cumulatively with the control commands of the acquisition computer of the acquisition system.

Within the scope of the invention, it is mandatory to execute the steps of the method that are mentioned in the preceding in the order that is described in the preceding. For example, it is alternatively possible to initially conclude the finding process and only then generate the control data at the finding system in order to initiate the transmission of modified image data. In a further embodiment, the method steps of importing the image data and registering control data can be interleaved with one another (interleaving) so that, given presentation of image data, a window can be immediately activated on the monitor of the finding system, via which window control data can be input with regard to the respectively presented images. This window can be activated either for an image or for a group of images (for example an image series of an examination), for instance via a mouse click.

Moreover, it is possible for individual segments of the method described in the preceding to be fashioned as individual commercial units, and remaining segments of the method can be fashioned as other commercial units. The method according to the invention can therefore be executed as a distributed system on different computer-based instance (for example client/server instances). For example, it is thus possible that the control module for its part comprises different sub-modules that are implemented in part at the acquisition system, in part at the finding system and/or in part at other computer-based instances.

The above object also is achieved by a non-transitory, computer-readable storage medium encoded with programming instructions are stored in a memory of a computer and are computer-readable commands that are designed to execute the method described in the preceding when the commands are executed on the computer. The commands also can be downloaded from a server via a corresponding network.

### BRIEF DESCRIPTION OF THE DRAWINGS

FIG. 1 is a schematic representation of acquisition system and finding system according to the prior art.

FIG. 2 is a schematic representation of a finding system expanded according to the invention, according to a preferred embodiment of the invention.

FIG. 3 is a flowchart of a preferred workflow of a method according to the invention.

FIG. 4 is a schematic representation of a finding system expanded according to the invention, with additional modules.

### DESCRIPTION OF THE PREFERRED EMBODIMENTS

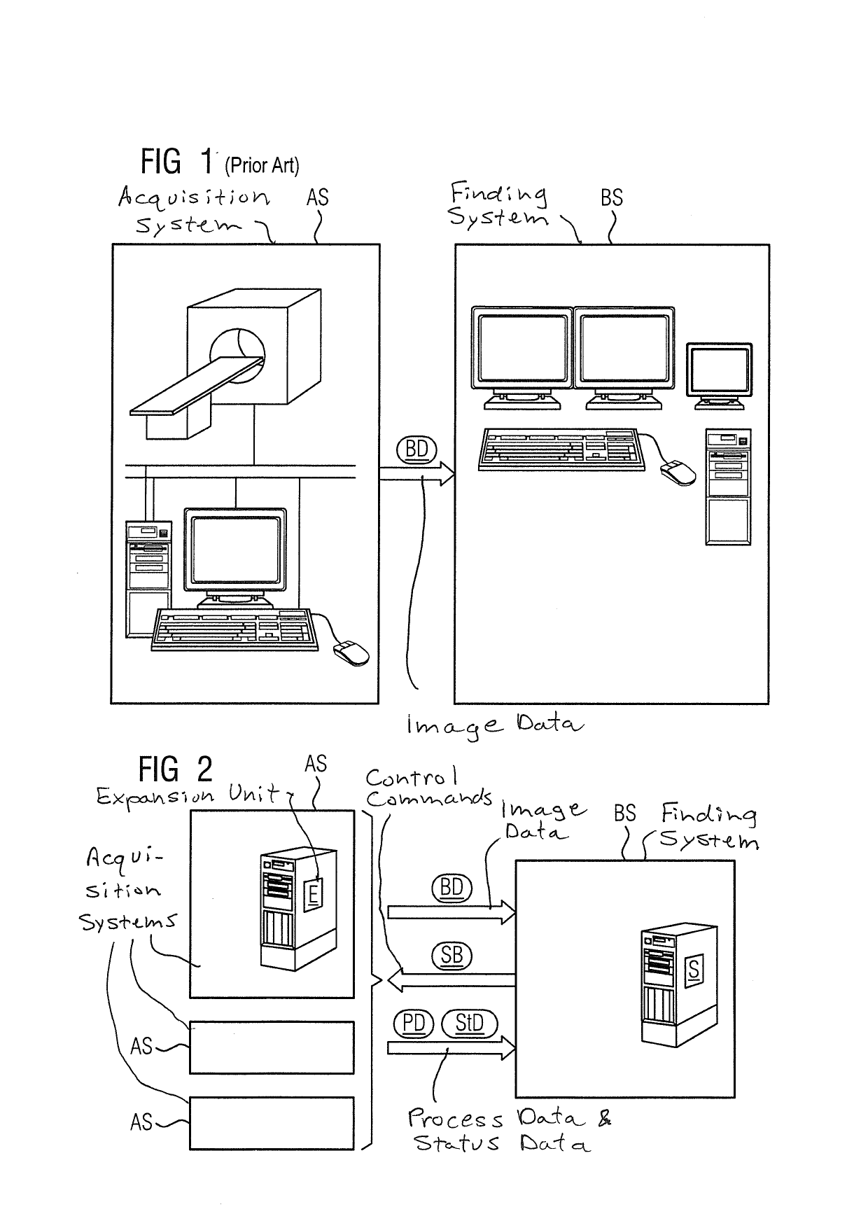

The invention and its differences relative to the prior art are explained in detail in the following using FIGS. 1 and 2.

A known infrastructure that is applied in the known systems in the prior art for assessing medical image data is schematically shown in FIG. 1. An acquisition system AS serves to acquire medical image data. In a preferred embodiment, this acquisition system AS comprises a magnetic resonance system that is connected via a network (local area network—LAN—or wide area network—WAN) with computer-based instances or consoles. Multiple computer workstations can also be provided here. A control panel is provided in order to control the acquisition process. Here the measurement sequences are specified (or are preconfigured) in order to adapt the scanner to the respective medical application case. These workstations are designated as “scanner workstations”. Here a measurement program for the MR scanner runs that comprises a sequence of different program steps. The generated medical image data can be presented via a software (graphical slice positioning software—GSP—for example) on a monitor. The generated image data BD are transmitted via a network to a remotely located finding system BS. The finding system BS possibly also includes multiple consoles in order to view the medical image data and create a finding from these. A radiologist can access image data with the aid of a PACS system.

As shown in FIG. 1, however, in the prior art it is not possible for the radiologist working at the finding workstation to directly affect the image data acquisition at the scanner workstation.

However, in the event that the radiologist at the finding system BS sees image data that cannot be used for creating the finding (for example because they are too blurry, cover a wrong region, or additional exposures are required etc.) in the previous known systems the radiologist could not directly take any measures from the finding workflow in order to modify the image data acquisition.

The present invention is applied here. It expands the finding system BS with a control module S. The acquisition system is advantageously also expanded by an expansion module E (or expansion unit). The acquisition system AS can also include multiple MR scanners. This is represented on the left side in FIG. 2 with the additional boxes that all bear the reference character AS. The acquisition systems AS serve to acquire image data BD that are transmitted to the finding system BS after conclusion of the acquisition process. Moreover, it is possible that the acquisition system additionally transmits status data StD and process data PD to the finding system BS in addition to the image data BD. The control module S of the finding system BS serves to register control data at the finding system BS. The control data are typically user inputs of the radiologist. The control data are converted into control commands SB in the control module S. The control commands are subsequently transmitted to the acquisition system AS for execution. The control commands SB are thereby designed such that they can be directly imported from the acquisition system and be used there to control the acquisition process. The specific conditions of the respective acquisition system AS are thus taken into account here (platform, operating system, compression requirements, data transfer rate etc.).

The transmitted control commands SB are advantageously an append command and/or a repeat command. The append command serves to produce an additional measurement sequence or additional measurements so that additional image data sets are generated and can be transmitted to the finding system BS for more detailed assessment. The repeat command serves to repeat an already executed measurement again. For example, this makes sense in the event that the patient could not hold his breath for a sufficient length of time, and therefore the exposure is blurry. It is likewise possible that the patient has moved during the acquisition duration. The same measurement should then be repeated again with the same measurement parameters. The image data BD that are thereby acquired are then in turn transmitted to the finding system BS for the purposes of assessment.

The expansion module E of the acquisition system AS is, so to speak, the counterpart of the control module S of the finding system BS and interacts with said control module S. The expansion module E serves to receive the control commands BS of the control module S, and furthermore likewise serves to transmit status data StD and process data PD from the acquisition system AS to the finding system BS. In an expanded embodiment, the expansion module E can also include additional functionalities and, for example, be designed to show the received control commands SB on a monitor or relay these to remote instances.

As shown in FIG. 2, the network connection between the acquisition system AS and the finding system BS is not a unimodal communication connection (as is the case in the prior art; see FIG. 1, in which exclusively the direction from the acquisition system AS to the finding system BS is provided), but rather is a bidirectional communication connection that enables a data exchange in both directions. In particular, image data can be transferred from the acquisition system AS to the finding workstation, and control commands SB can be transferred from the finding workflow directly to the acquisition system AS to control the acquisition. In the previous system in the prior art—for example in the systems with the syngo Expert-i software—a remote access with complete functionality of the scanner workstation is provided, which requires additional authorization measures. These can advantageously be avoided with the solution according to the invention since only preselected control commands (in particular only the append command and the repeat command) should be executed at the scanner workstation.

In a schematic presentation, FIG. 4 shows additional modules of the finding system BS that has been expanded with the control module S. The control module S includes a converter. The converter K is designed to convert the control data registered locally at the finding system BS into control commands SB. The converter K operates wholly automatically after it has received the control data via a user interface. The control data can be entered as an input in the form of, for example, a mouse-controlled selection of a list point of a list menu that is presented at the interface of the finding software on a finding monitor M.

In an embodiment, the control module S can include a transformation unit T. The transformation unit T serves to send message packets to the acquisition system AS and to receive message packets from the acquisition system AS. The message packets are thereby automatically adapted to the information technology requirements (data exchange, platform of finding system and/or acquisition system etc.). A significant advantage of the achievement according to the invention is that the control commands SB of the control module S are transmitted via the same interface as the image data BD that are sent from the acquisition system AS to the finding system BS. A design of an additional communication interface is unnecessary.

An additional characteristic of the achievement of the object according to the invention is that the control method according to the invention can be executed in a single stage. In other words, an action can be introduced directly from the finding workflow to the acquisition workstation AS with a user action (for example a mouse click to select a control command SB, in particular an append/repeat instruction). In other words, within the scope of the finding at the finding workstation BS control instructions to control the image acquisition at the acquisition system AS can be directly executed without needing to leave the finding workflow or without additional communication channels or communication steps being required (for example in that the radiologist at the finding system BS contacts the MTA at the scanner workstation AS, for example via telephone).

According to the invention, it is provided that the images of the patient that are to be assessed are displayed at the monitor M of the finding system BS in what is known as a syngo.via Reading Task. The radiologist can now view the image data. In the event that it turns out that one or more images cannot be used for the finding, the radiologist can call up the control commands BS, for example via a context menu in the displayed image for example, it can be preset that a context menu that includes different entries appears upon clicking a mouse button (right or left mouse button) on the respective image. The entries in particular include a repeat function and an append instruction. In further embodiments, here additional entries can be included that, for example, relate to functions for the finding, for example: a copy function; a delete function; a load series function; or functions to display additional data sets with regard to the image data to be assessed. These can deal with properties (for example “Show Properties”) that are relevant within the scope of the acquisition of the image data, or with data with regard to the acquisition protocol (“View Protocol”, for example). In an alternative embodiment of the invention, this context menu with the selectable control instructions SB is not directly initiated from the respective image but rather within a work step of the finding workflow. For example, the work step can also be situated before presentation of the respective image data and after calling the finding software.

The transformation unit T of the control module S is designed to transmit the control command SB (selected by means of the shown context menu, for example) to the scanner workstation. If the command pertains to the repeat command, the identifiers of the respective image are transmitted as an additional parameter. It is therefore ensured that the acquisition system AS also unambiguously knows the association with the respective image that should be measured again. The identifier is one-to-one in the mathematical sense, such that a bijective relation is provided between image and identifier. Via the identifier the respective image can be identified and located both in a database of the scanner (not shown further in Figures) and in the database DB of the finding workstation BS.

According to a preferred embodiment, the control commands SB are transferred via a web service. This web service is normally based on xml-based messages and a data exchange via Internet-based protocols that are typically built on a service-oriented architecture (SOA).

According to a further aspect of the invention, the application of what are known as Remote Procedure Calls (RPC) is resorted to for the implementation of the interprocess communication between acquisition system AS on the one hand and finding system BS on the other hand. The communication via RPC calls can be directly executed bidirectionally and has the advantage that a client/server model can be implemented between the otherwise separate acquisition system AS and finding system BS. An RPC-based interprocess communication is offered by Microsoft (“.NET Remoting”, for example) or Sun, among others. In a preferred embodiment of the invention, the finding system BS sends additional job data to the acquisition system AS with the message packet that includes the control commands SB. The additional job data are, for example, the identifier of the respective image at which the command should be executed. Optionally, additional job data can be transmitted here, for example: after processing the job query at the acquisition system AS via execution of the requested MR measurement, the acquisition system AS sends a job response as a message back to the finding system BS. The job response can be divided into multiple message packets and can in particular include the modified or newly generated image data BD. Moreover, it is possible to also send status data StD and process data PD to the finding system BS in a separate response message. These can be presented on the monitor M of the finding system BS. The assessor at the finding workstation can therefore receive an overview of the MR additional measurements that he commissioned.

In an embodiment of the invention, the process data PD are sent from the acquisition system AS to the finding system BS independently of the transmission of the control commands SB. The process data PD in principle relate to the processing of the respective MR measurement for the image data that should be viewed at the finding workstation. The assessing radiologist must be able to determine which state the MR measurement occurred. This is therefore necessary since the finding workstation is normally located separate from the acquisition system AS, and the radiologist thus has no visual contact with the scanner. The radiologist therefore may not know to what extent the execution of the measurement program has already proceeded, whether the patient has already received an injection of contrast agent, and/or whether the patient is still located on the MR table. This information is important in order to know whether specific control commands SB can still be executed at all. The process data PD thus serve as background and auxiliary information to control the acquisition process at the finding system BS. In order to ensure that only relevant data are displayed at the finding system BS, in an advantageous development it is provided that the process data PD are shown specific to the situation. In particular, it is provided that the status data StD and/or the process data PD are only shown (at the finding system BS) in the event that the user requests the input of control commands SB.

In a further embodiment of the present invention, a chat functionality or, respectively, a corresponding chat module (not shown in Figures) is provided. The chat module is designed to exchange additional message packets between finding system BS and acquisition system AS. Length-limited text inputs should thereby be transmitted immediately to the respective partner system. An example of an implementation is described in the following:

A window in which the radiologist can input questions and/or instructions for the acquisition system AS appears on the monitor M of the finding workstation BS, in spatial proximity to the button for a control command SB (for example repeat function or append function). For example, here the radiologist could input: “Please don't let the patient go yet.” After inputting this text message, a send button can be clickable that initiates the transmission of this text message to the scanner AS. After sending this text message, the user at the acquisition system AS can confirm the receipt of this text message. It is likewise possible that the transmission of text messages can also be initiated at the acquisition system AS. It is thus possible that the questions of the finding workstation BS are answered at the acquisition system AS and transmitted back.

In order to not interfere with the workflow of the image acquisition at the acquisition system AS, according to an advantageous development of the invention it is provided that the user of the acquisition system AS receives a veto right regarding the execution of the control commands SB that have been initiated at the control module S. For example, in the event that a contrast agent injection of the patient to be examined is already far in the past, a current execution of a repeat command no longer makes since a wash-out effect of the contrast agent has already occurred and a sufficient contrast agent enrichment can no longer be ensured. In this case, the MTA at the scanner workstation AS can reject the execution of the control commands SB. The MTA additionally receives the possibility to send a corresponding text message (including a reason for the rejection, for example) to the finding system BS.

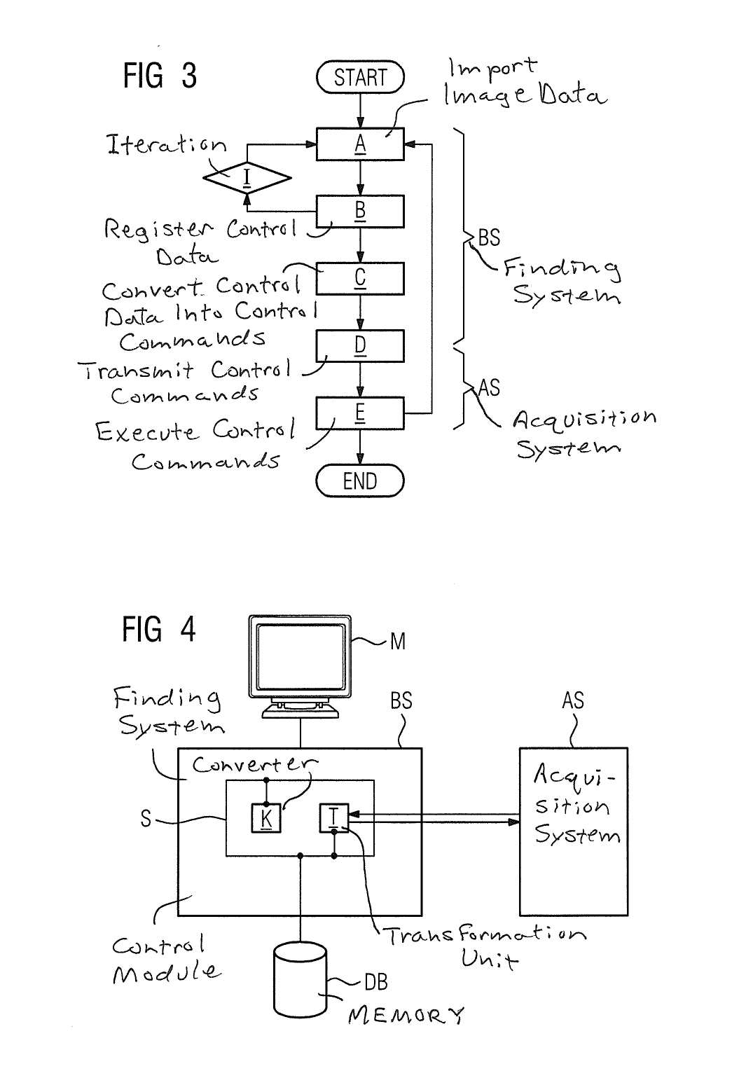

In the following a workflow according to a preferred embodiment is described in detail with reference to FIG. 3.

After the start of the system, in Step A the image data of the acquisition system AS are imported at the finding computer of the finding system BS. For this the data are imported via the provided interface between acquisition system AS and finding system BS. The message exchange can thereby optionally be initiated by the acquisition system AS or by the finding system BS.

The registration of control data at the finding system BS takes place in Step B. This step is naturally optional and is only executed when control data are actually input at the interface of the finding system via a user interface. The step characterized with reference character I in FIG. 3 represents one possible iteration of Steps A and B. This means that, according to a first embodiment variant, it is preset to initially present all image data of an image series to be assessed and to register control data in connection with this. In an alternative embodiment, control data can be input at the interface of the finding system BS at any time.

The control data are advantageously input via the selection of an entry of a context menu. In this case the radiologist can click on the entry “Append Command” and/or on the entry “Repeat Command”. The selected entries therefore apply as control data and are input at the finding system BS.

A conversion of these control data into control commands SB is subsequently executed in Step C. The control commands SW are specific to the acquisition system and can be directly imported by this and be executed.

The control commands SB can be transmitted to the acquisition system in method Step D.

Following this is the method step E, which executes the control commands SB at the acquisition system AS and therefore initiates a modified control of the image acquisition.

As shown in FIG. 3, Steps A through and including C are advantageously executed at the finding system BS, while Step E is executed at the acquisition system AS. Step D is executed in part at the finding system BS and in part at the acquisition system AS. The method ends after execution of Step E.

The invention can be briefly summarized as follows: the invention implements a direct network connection between the otherwise separately operating acquisition system AS and finding system BS. Furthermore, according to the invention an integrated interprocess communication can be provided between acquisition and finding at the finding system BS.

A series of advantages can be achieved via the solution according to the invention. On the one hand it is possible to provide control commands SB that are preconfigured within the finding workflow to control the image acquisition process. These control commands SB can be transmitted directly and automatically to the acquisition system AS for execution (thus for control) without additional authorization measures and/or other communication channels needing to be activated. This has the advantage that the finding physician can initiate control measures at the remote acquisition system AS directly from his workstation, without needing to leave his workstation or call other applications. This leads overall to a higher finding quality, to a more efficient finding (time savings), and increases the operating comfort.

Finally, it is noted that the invention with the exemplary embodiments is not limited with regard to a specific physical realization of the invention. For example, the invention is not limited to MR measurements, but rather can also be similarly used for other imaging systems. Moreover, it is also not absolutely necessary to resort to a SUA-, RPC- and/or web service-based communication technology. For example, proprietary protocols can also be used for process communication. Moreover, the invention can be implemented partially or wholly in software and/or in hardware. Moreover, the method and the control system according to the invention can be realized distributed on multiple physical products (comprising computer program products). It is thus possible to implement a portion of the control at the finding system BS and a remaining portion of the control at the acquisition system AS.

Although modifications and changes may be suggested by those skilled in the art, it is the intention of the inventors to embody within the patent warranted hereon all changes and modifications as reasonably and properly come within the scope of their contribution to the art.

We claim as our invention:

1. A computerized medical finding system to assist medical images, comprising:
a computer;
a monitor in communication with said computer;
a user interface in communication with said computer;
said computer comprising an acquisition system interface configured to place said computer in communication with a medical image data acquisition system that is located remote from said computer, in order to import into said computer, from said medical image data acquisition system, image data acquired by operation of said medical image data acquisition system operated according to first control commands executed by a control unit of said medical image data acquisition system;
said computer being configured to display a medical image at said monitor corresponding to said medical image data;
said computer being configured to receive control data, entered into said computer via said user interface, that describe operation of said medical image data acquisition system to acquire new medical image data that will produce a new medical image that differs from the medical image displayed at said monitor;
said computer being configured to automatically convert said control data into new control commands in a form executable by said control unit of said medical image data acquisition system, and to transmit said new control commands directly and automatically to said medical image data acquisition system via said acquisition system interface; and
said computer being configured to receive status data, via said acquisition system interface, generated by said image data acquisition system during execution of said new control commands, and to display said status data at said monitor.

2. A computerized medical finding system as claimed in claim 1 wherein said computer is configured to convert said control data into control commands that include control commands selected from the group consisting of an append command, a repeat command, and an additional control command that is not included in said first control commands.

3. A computerized medical finding system as claimed in claim 1 wherein said computer is configured to automatically register said control data and to integrate said control data into a finding procedure.

4. A computerized medical finding system as claimed in claim 1 wherein said computer is configured to receive an input via said user interface that causes said control data to be registered and integrated into a finding procedure.

5. A computerized medical finding system as claimed in claim 1 wherein said control module is configured to cause said control unit of said medical image data acquisition system to execute said new control commands without a separate additional registration of said control data.

6. A computerized medical finding system as claimed in claim 1 wherein said control module is configured to cause operation of said medical image data acquisition system without a confirmation signal being required at said medical image data acquisition system.

7. A computerized medical finding system as claimed in claim 1 wherein said control module is configured to cause said control unit to execute said new control commands at a time that is decoupled from at least one of transmission of said new control commands on said control module and receipt of the transmitted control commands at said medical image data acquisition system.

8. A computerized medical finding system as claimed in claim 1 wherein said control module is configured, via said acquisition system interface, to participate in an inter-process communication with said medical image data acquisition system via a web service, in order to receive said image data or to transmit said new control commands.

9. A system for acquiring and assessing medical image data, comprising:
a medical image data acquisition apparatus comprising a control unit;
a computer located remote from said medical image data acquisition apparatus;
a monitor in communication with said computer;
a user interface in communication with said computer;
said computer comprising an acquisition system interface configured to place said computer in communication with said medical image data acquisition apparatus in order to import into said computer, from said medical image data acquisition system, image data acquired by operation of said medical image data acquisition apparatus operated according to first control commands executed by said control unit of said medical image data acquisition system;
said computer being configured to display a medical image at said monitor corresponding to said medical image data;
said computer being configured to receive control data, entered into said computer via said user interface, that describe operation of said medical image data acquisition apparatus to acquire new medical image data that will produce a new medical image that differs from the medical image displayed at said monitor;
said computer being configured to automatically convert said control data into new control commands in a form executable by said control unit of said medical image data acquisition apparatus, and to transmit said new control commands directly and automatically to said medical image data acquisition apparatus via said acquisition system interface; and
said computer being configured to receive status data, via said acquisition system interface, generated by said image data acquisition apparatus during execution of said new control commands, and to display said status data at said monitor.

10. A method for acquiring and assessing medical image data, comprising:
providing a computer at a location remote from a medical image data acquisition system, said computer having a monitor in communication with said computer, and a user interface in communication with said computer;
via an acquisition system interface, placing said computer in communication with said medical image data acquisition system and importing, into said computer, from said medical image data acquisition system, image data acquired by operation of said medical image data acquisition system operated according to first control commands executed by a control unit of said medical image data acquisition system;
from said computer, displaying a medical image at said monitor corresponding to said medical image data;
at said computer, receiving control data, entered into said computer via said user interface, that describe operation of said medical image data acquisition system to acquire new medical image data that will produce a new medical image that differs from the medical image displayed at said monitor;
in said computer, converting said control data into new control commands in a form executable by said control unit of said medical image data acquisition system, and transmitting said new control commands directly and automatically to said medical image data acquisition system via said acquisition system interface; and
at said computer, receiving status data, via said acquisition system interface, generated by said image data acquisition system during execution of said new control commands, and displaying said status data at said monitor.

11. A non-transitory, computer-readable data storage medium encoded with programming instructions, said data storage medium being loaded into a control unit of a medical image data acquisition system and a control module of a computerized medical finding system located remote from medical image data acquisition system, said medical image finding system comprising a computer in which said control module is contained, and a monitor and a user interface in communication with said computer, and said computer comprising an acquisition system interface, said programming instructions causing said computer to:
communicate with said medical image data acquisition system via said acquisition system interface, in order to import into said computer, from said medical image data acquisition system, image data acquired by operation of said medical image data acquisition system operated according to first control commands executed by a control unit of said medical image data acquisition system;
display a medical image at said monitor corresponding to said medical image data;
receive control data, entered into said computer via said user interface, that describe operation of said medical image data acquisition system to acquire new medical image data that will produce a new medical image that differs from the medical image displayed at said monitor;
automatically convert said control data into new control commands in a form executable by said control unit of said medical image data acquisition system, and to transmit said new control commands directly and automatically to said medical image data acquisition system via said acquisition system interface; and
receive status data, via said acquisition system interface, generated by said image data acquisition system during execution of said new control commands, and display said status data at said monitor.
